(19) United States
(12) Patent Application Publication
(10) Pub. No.: US 2014/0276188 A1
(43) Pub. Date: Sep. 18, 2014

(54) SYSTEMS, METHODS AND DEVICES FOR ASSESSING AND TREATING PAIN, DISCOMFORT AND ANXIETY

(71) Applicant: ACCENDOWAVE INC., POWAY, CA (US)

(72) Inventor: Cary Jardin, Poway, CA (US)

(73) Assignee: ACCENDOWAVE INC., POWAY, CA (US)

(21) Appl. No.: 14/209,615

(22) Filed: Mar. 13, 2014

Related U.S. Application Data
(60) Provisional application No. 61/785,693, filed on Mar. 14, 2013.

(51) Int. Cl. A61B 5/04; A61B 5/00

(52) U.S. Cl. CPC A61B 5/04012; A61B 5/4824
USPC 600/544

(57) ABSTRACT

Systems, methods and devices are provided for assessing and/or treating pain, discomfort, and/or anxiety through the development of an algorithm based on a correlation between electroencephalography (EEG) signals received from a patient and the patient's self-assessed levels of pain. The pain detection algorithm is then applied to EEG signals obtained from any patient and used to assess the patient's level of pain without requiring other input from the patient. One or more devices in a system may be implemented to measure the EEG signals, apply the algorithm and display a level of pain in real-time to a patient or healthcare provider. The real-time pain assessment may be used to continuously monitor whether a patient is in pain and select one or more treatments to minimize the patient's perception of pain and avoid prescribing unnecessary medications.

### CROSS-REFERENCE TO RELATED APPLICATIONS

This application claims the benefit of U.S. provisional app. Ser. No. 61/785,693, filed on Mar. 14, 2013, which is hereby incorporated by reference.

### BACKGROUND

1. Field

The subject matter discussed herein relates to detecting and treating pain in a human or animal, and more specifically to systems, methods and devices for assessing levels of pain, discomfort or anxiety in real-time and applying a treatment to reduce the level of pain without medication.

2. Related Background

Determining the amount of pain that a person or animal is experiencing remains a difficult task. In the healthcare, medical and veterinary fields, understanding and minimizing pain is an important goal. However, current methods of pain assessment continue to rely on verbal feedback from the patient as to whether they feel pain and how much pain they are experiencing. Patients are often asked to rate the level of pain they are experiencing based on a simple numerical rating system, where each number corresponds to a level of pain. In some instances, the rating system also includes illustrations of cartoon facial expressions where the facial expression is supposedly indicative of a corresponding level of pain. This rating system is subjective and lacks any empirical data to support the rating provided. A patient may not be able to match their pain to a facial expression with any accuracy, and the patient may also deliberately lie about their level of pain in order to obtain pain medication from a doctor.

Self-assessment of pain levels is also not possible on patients that are unable to communicate—whether the patient is an infant, a person who is unconscious or an animal. For non-communicative patients, a healthcare provider is left to their own abilities to assess whether the patient is experiencing pain.

In addition to assessing pain, efforts are also being made to assess discomfort, stress or anxiety and help a patient manage these feelings without the need for medication or more involved therapies. However, assessing these other feelings is also primarily subjective and determined almost solely by a self-assessment from the patient, or at best, a limited assessment of the patient's mental and physical state by a healthcare provider.

### SUMMARY

Embodiments described herein are directed to systems, methods and devices for assessing and treating pain, discomfort and anxiety through the development of an algorithm based on a correlation between electroencephalography (EEG) signals received from a patient and the patient's self-assessed levels of pain. The pain detection algorithm is then applied to EEG signals obtained from any patient and used to automatically assess the patient's level of pain without requiring other input from the patient. One or more devices in a system may be implemented to measure the EEG signals, apply the algorithm and display a level of pain in real-time to a patient or healthcare provider. The real-time pain assessment may be used to continuously monitor whether a patient is in pain and select one or more treatments to minimize the patient's perception of pain and avoid prescribing unnecessary medications.

In some embodiments, a method of assessing pain in a patient comprises the steps of: obtaining at least one electroencephalography (EEG) signal from a patient; measuring levels of alpha waves in the EEG signal over a period of time; measuring levels of beta waves in the EEG signal over the period of time; correlating the measured levels of alpha waves and the measured levels of beta waves with an application of a negative external stimulus over the period of time; training an algorithm using a computer with a processor and a memory to analyze EEG signals and predict when a patient is experiencing pain based on the correlated negative external stimulus and levels of alpha waves and beta waves; and outputting the trained algorithm (a pain detection algorithm trained to detect pain levels).

Additional aspects related to the subject matter discussed herein will be set forth in part in the description which follows, and in part will be apparent from the description, or may be learned by practice of the subject matter. Aspects of the subject matter discussed herein may be realized and attained by means of the elements and combinations of various elements and aspects particularly pointed out in the following detailed description and the appended claims.

It is to be understood that both the foregoing and the following descriptions are exemplary and explanatory only and are not intended to limit the subject matter described herein in any manner whatsoever.

### BRIEF DESCRIPTION OF THE DRAWINGS

FIG. 1 is a functional block diagram illustrating an example system for assessing a level of pain in a patient, according to some embodiments.

FIG. 2 is a flowchart illustrating an example method of collecting data for developing a pain detection algorithm, according to some embodiments.

FIG. 3 is a flowchart illustrating an example method of correlating data for developing the pain detection algorithm and applying the algorithm to data to assess a level of pain in a patient, according to some embodiments.

FIG. 4 is a flowchart illustrating an example correlation of electroencephalography (EEG) signals with the experiencing of pain or mental relaxation, according to some embodiments.

FIG. 5A illustrates an example device for detecting and wirelessly transmitting EEG signals, according to some embodiments.

FIG. 5B illustrates an example device being used for detecting and wirelessly transmitting EEG signals, according to some embodiments.

FIG. 6 illustrates an example device which is connected with the headset device for receiving the EEG signals and assessing a level of pain in a patient for display on the mobile device, according to some embodiments.

FIG. 7 illustrates a visual display of the EEG signals, according to some embodiments.

FIG. 8 illustrates an example pain feedback device with a plurality of pain indicator ears, according to some embodiments.

FIG. 9 illustrates different example positions of the pain indicator ears of FIG. 8 as they correspond to different levels of pain, according to some embodiments.

FIG. 10 illustrates the different example positions of the pain indicator ears of FIG. 9 as they correspond to varying levels of mental stress, relaxation and pain, according to some embodiments.

FIG. 11 shows an example of a process implementation.

FIG. 12 shows an example computing environment with an example computing device suitable for use in some example implementations.

### DETAILED DESCRIPTION

The subject matter described herein is taught by way of example implementations. Various details have been omitted for the sake of clarity and to avoid obscuring the subject matter. The examples shown below are directed to structures and functions for implementing systems, methods, and/or devices for assessing and/or treating pain, discomfort, and/or anxiety.

In the following description, reference will be made to the accompanying drawings. The aforementioned accompanying drawings show by way of illustration, and not by way of limitation, specific embodiments and implementations consistent with principles of the subject matter described herein. These implementations are described in sufficient detail to enable those skilled in the art to practice the subject matter, and it is to be understood that other implementations may be utilized and that structural changes and/or substitutions of various elements may be made without departing from the scope and spirit of subject matter described herein. The following detailed description is, therefore, not to be construed in a limited sense. Additionally, the various embodiments of the subject matter discussed herein may be implemented in the form of software running on a general purpose computer, in the form of a specialized hardware, or combination of software and hardware.

Described below are systems, methods and devices for assessing and treating pain, discomfort and anxiety (collectively referred to as “pain” or “pain level”). In a first section, a description is provided of the development of a pain detection algorithm using measurements of electroencephalography (EEG) signals and correlations with self-assessed pain levels and pain device application levels. The pain detection algorithm is then applied to new EEG signal data to automatically assess pain levels without input from the patient. In a second section, the assessed pain level may be utilized to monitor and provide treatments to minimize the patient's perception of pain.

I. Measuring and Assessing Pain, Discomfort and Anxiety

Measuring and assessing pain, discomfort and anxiety may be achieved more empirically through the use of physiological measurements of a patient, such as with electroencephalography (EEG) signals or computer-aided visual analysis of facial expressions. As will be described in further detail below, these physiological measurements may be used to develop a pain detection algorithm which can then be applied to EEG signals from a patient to immediately assess the patient's level of pain in real-time. Devices for measuring the EEG signals, computing the correlations and displaying the measured levels of pain may also be provided.

Pain Assessment System

FIG. 1 is a functional block diagram illustrating an example system for assessing a level of pain in a patient, according to some embodiments. An EEG sensor 102, with one or more electrodes or detection points, captures EEG signals from a patient (e.g., any living thing that produces EEG signals, such as a human being or an animal that is not a human being), which are then transmitted (either wired or wirelessly) to a computing device for analysis, such as a mobile device 104 or a server 106, or both. Mobile device 104 is for discussion only. In actual implementations, mobile device 104 may refer to any computing device capable of performing the described functions, mobile or not mobile. The EEG signals are electrical charges detected from one or more locations on the patient's body, often the patient's head. The electrical charges may be studied by reviewing the frequency of activity of a charge—i.e. the frequency waves—which may be visualized and correlated to brain activity. In some embodiments, a single input for each EEG frequency from 1 Hz to 200 Hz may be used. An additional input corresponding to a slope of, for example, the last 10 seconds of each EEG frequency from 1 Hz to 200 Hz may also be used. The 10 seconds or another time window of frequency data may be used to perform a straight line fit and compute a slope and/or a trend for each frequency.

In some implementation, additional inputs, such as an input corresponding to facial movement may also be used to gather EEG data. Facial movement has high intensity electrical burst in the EEG circuit. The electrical burst triggers the muscle movement. In addition to detecting EEG signals from general facial movement, some implementations may detect EEG signals from specific facial moment, such as eye movement. For example, the electrical burst associated with eye movement may be processed (e.g., using facial recognition) in conjunction with facial images or video captured using a camera of a device (e.g., mobile device 104).

The mobile device 104 or server 106 may store the EEG data locally on resident memory, or they may store the EEG data on a separate database such as the database 108. The mobile device 104 or server 106 may use the EEG data to compute a pain detection algorithm, as described in detail below, and apply the algorithm to new EEG data to generate reports on assessed levels of pain. The mobile device 104 or server 106 may also activate applications that alert, for example, a healthcare provider of the assessed level of pain and/or provide pain management therapies for the patient through one or more applications running on the mobile device 104 and/or server 106. A report or data on the assessed level of pain and/or an alert may be generated for a healthcare provider, which may be transmitted to a healthcare device 110 where it can be displayed to a doctor, nurse, veterinarian or other medical or healthcare professional who is tasked with monitoring the patient's level of pain.

Pain Assessment Algorithms

Using the example system 100 described above, the EEG signals obtained from the patient are first used in conjunction with additional input data to generate a pain detection algorithm. In developing the pain detection algorithm, the input data is known, the output (pain level from the pain expression device) is known, but the relationships and correlation of the input data that result in the pain level is not known. Therefore, the process of developing the pain assessment algorithm looks to mathematical correlations and learning machines to translate the input sets of numbers into a single value which corresponds to a level of pain, discomfort or anxiety. Therefore, when the patient is asked to indicate the level of pain they are experiencing (using the pain expression device), a comparison can be made with the computed pain level to determine if the algorithm has been accurately configured.

FIG. 2 is a flowchart illustrating an example method of collecting data for developing a pain detection algorithm, according to some embodiments. In a first step 210, an EEG sensor is attached to a patient, while in a second step 220, the patient is provided with a pain expression device, such as a squeeze ball or other pressure sensitive device which can measure the amount of force applied. The patient will be instructed to apply an amount of force to the pain expression device that they believe corresponds to the amount of pain they are experiencing. A pain administrating device may be used to manually apply pain to the patient, such as a tattoo needle, and, at 230, a sensor may also be attached to the pain administrating device in order to measure the amount of force being applied by the pain administering device so that it can be correlated with the EEG signals and the patient's reaction on the pain expression device. The sensor on the pain administration device may be an optional component, but may help to determine “pre-pain” mental stress when the data from the EEG and pain expression device are correlated.

At 240, a computing device such as the mobile device 104 or server 106 may then receive the data from the EEG sensor, pain expression device and pain administering device and store the data. At 250, the computing device will then synchronize the sources of data based on the time they were collected in order to prepare for the correlation of the data in the following step.

A pain detection algorithm is an algorithm trained for detecting pain levels (e.g., the algorithm can be executed or followed by a machine or computing device to detect pain levels). A pain detection algorithm can be trained using, for example, machine learning. For example, an algorithm can be trained using available techniques known as support vector machine and/or artificial neural network. Other techniques may be employed to create a trained algorithm or pain detection algorithm.

One or more pain detection algorithms may be created specific for detecting pain levels of an individual patient. Pain detection algorithms may be created for different individual patients.

One or more pain detection algorithms may be created for detecting pain levels of patients in general (e.g., the algorithm may be trained for used on certain segments of patients or all patients). For example, EEG signals of a sample group of patients may be used to train or created a pain detection algorithm (general pain detection algorithm). When the general pain detection algorithm reaches a threshold certainty (e.g., can detect pain levels to X percentage of accuracy), the general pain detection algorithm can be used to detect pain levels of patients what are not in the sample group of patients.

The threshold certainty can be determined, for example, by testing the general pain detection algorithm against individual pain detection algorithms with patients who are in the sample group. A general pain detection algorithm may be continued to trained (e.g., using EEG signals from more patients) until it has achieved a certain level of confidence or certainty.

In algorithm training, once the data has been captured, it is correlated and processed to develop a pain detection algorithm. In some embodiments, the input data is weighted before summing the data in order to alter the importance of one or more inputs. In another embodiment, each input may also have a transfer function applied, wherein an output is generated only when a certain threshold value is reached. By applying selected weights and transfer functions, a desired output pain assessment level is achieved which matches that provided by the patient from the pain expression device.

One example method to find the weights and transfer functions that will produce the desired output is that the problem space may be searched or selected by randomly guessing combinations of weights and transfer functions and selecting the best fits. For example, using support vector machine, artificial neural network, and/or other techniques, the computed output value (e.g., the correlation of the input EEG signals with a pain level) is checked against the provided input (e.g., the input EEG signals and the stimulated pain level). The error between the computed output and the provided input is computed, then the weight that most contributed to the error is adjusted and the system is re-run. In this way, the computed output is used to reverse propagate the error into the system. The system learns by reducing the errors of the weights that produced computed output. The computed output is accepted when the error is at a certain level, which is different and adjustable according to implementations.

This example method may be repeated one or more times using different provided inputs to generated different computed outputs. The characteristics of the EEG signals of a set of provided input may be analyzed using alpha waves, beta waves, and/or other waves.

The best fit combinations can then be further processed into further iterations of searches in order to find better fits for the data which will eventually result in an algorithm which satisfies a threshold confidence. Searching through the problem space may be carried out with various different computer learning methods, including genetic algorithms, support vector machines or artificial neural networks. Other methods may be implemented to produce the desired output.

FIG. 3 is a flowchart illustrating an example method of correlating data for developing the pain detection algorithm and applying the algorithm to data to assess a level of pain in a patient, according to some embodiments. The EEG data 310, pain expression data 320, and (optionally) pain administering data 330 are correlated and processed using, for example, a machine learning technique such as an artificial neural network, computer learning algorithm and/or support vector machine 340.

In some embodiments, both a genetic algorithm and a support vector machine, either individually or in combination, may be used to search and correlate input to the desired output. Both may be used due to the common problem of over optimizing the weights and transfer functions that will result in the system memorizing a training set.

The correlated data or trained algorithm may be used to assess and/or treating pain, discomfort, and/or anxiety. For example, feeding live EEG data 250 (e.g., live EEG signals detected from a person or animal) into a trained algorithm 360 (e.g., an algorithm that uses correlated data) will produce a computed pain level 370 based on the live EEG data. The pain level 370 may be logged, displayed, and/or shared (e.g., transmitted to a health care provider) for viewing, assessing, and/or treating (e.g., mitigating) the pain level.

FIG. 4 is a flowchart illustrating an example correlation of electroencephalography (EEG) signals with the experiencing of pain or mental relaxation, according to some embodiments. The patient's experience of pain can be correlated with the presence of alpha brain waves 410 and beta brain waves 415 in varying amounts. Alpha waves 410 are produced in conjunction with mental relaxation, or sleep 420, while beta waves (specifically low amplitude beta waves with multiple and low varying frequencies) are often associated with active, busy or anxious thinking, and active concentration 425. Thus, pain can be expressed as the lack of mental relaxation (e.g., low alpha waves). By finding a baseline of sampled data and known non-pain readings, average alpha and beta wave densities can be correlated to provide a “normal” state for a patient 430.

As a result of the correlation between alpha and beta waves, a determination can be made that average or above average alpha waves over a period of time is likely to indicate mental relaxation, and the lack of pain 440. A sustained absence of alpha waves over a specified period of time may indicate the possibility of pain 450, while a quick rise of beta waves over a specified time window may indicate mental stress and “breakthrough” levels of pain 460.

In some embodiments, a method of assessing pain in a patient may include the operations of: obtaining at least one electroencephalography (EEG) signal from a patient; measuring levels of alpha waves in the EEG signal over a period of time; in addition to or instead of measuring alpha waves, measuring levels of beta waves in the EEG signal over a period of time; correlating the measured levels of alpha waves and/or the measured levels of beta waves with an application of a negative external stimulus over the period of time (e.g., poking the patient with a needle, etc.); training an algorithm using a computer with a processor and a memory to analyze EEG signals and predict when a patient is experiencing pain based on the correlated negative external stimulus and levels of alpha waves and beta waves; and outputting the trained algorithm (e.g., an algorithm that uses correlated data to compute a pain level based on EEG signals). The trained algorithm may then be implemented or executed on a device (e.g., a mobile device) to assess and/or treat pain, discomfort, and/or anxiety. There are other types of brain waves, such as gamma, delta, mu and theta waves. Any one or more of these types of brain waves may be included or used in assessing pain.

As described above, an algorithm can be trained for use on a specific patient (personalized algorithm) or on a certain group of patients or all patients (general algorithm). An algorithm can be tuned or tweaked, for example, for use on another patient or another group of patients. For example, using an algorithm that has been trained to produce results closet to the desired results, the algorithm can be tuned in various ways, such as adjusting one or more correlated pain levels and/or further trained the algorithm with input signals from the target patient or patient group.

In general, for a general pain detection algorithm, the higher the amount of patient data are used to train or create the algorithm, the more stable and accurate the algorithm becomes. An algorithm created using breadth of data can be used on a more general group of patients (e.g., for making more general detections and decisions).

In some examples, a pain assessment method may be implemented with different, fewer, or more operations. The assessment method may be implemented as computer executable instructions, which can be stored on a medium, loaded onto one or more processors of one or more computing devices, and executed as a computer-implemented method.

Facial Expression Pain Assessments

In some embodiments, pain assessments may be determined through facial expressions, either by measuring EEG signals produced by changing facial expressions or by visual analysis of facial expressions captured by a camera facing the patient. In the EEG model, the muscles in a patient's face produce distinct EEG signals, which can then be associated with specific facial expressions that are indicative of the experiencing of pain. Pain assessments through facial expressions may be used separately or in conjunction with the EEG data described above.

Pain Assessment Devices

In some embodiments, various devices may be utilized to measure EEG signals from the patient. FIGS. 5A and 5B illustrate an example device for detecting and wirelessly transmitting EEG signals, according to some embodiments. The example device, such as a headset, may include one or more sensors, electrodes or detection points which are designed to be in contact with an EEG signal producer (e.g., a person or animal). The device may be referred to as an EEG sensor, which may have one or more sensors, electrodes, or detection points. Signals captured by the headset can be transmitted, wirelessly and/or by a wired connection, to a device such as the mobile device 102, where the received signals or data can be stored, processed, displayed (e.g., to the user for review), retransmitted, etc.

In some embodiments, a portable pain assessment device with a single (e.g., removable) sensor may be utilized, similar to a stethoscope, so that a healthcare professional can temporarily monitor a patient's EEG signals and then easily remove the sensor after a period of time.

One or more applications for monitoring and utilizing the pain assessment levels and the captured data may be provided in order to better diagnose and treat a patient's pain, discomfort or anxiety. FIG. 6 illustrates an example device which, as described above, may be connected with the headset device for receiving the EEG signals and computing a level of pain in a patient for display on the mobile device. The example device may be a mobile device or a device that is not generally considered as a mobile device. An application running on the device may provide detailed data to the user, such as a raw EEG signal, as illustrated in FIG. 7 (EEG signals displayed on a display screen). Additionally, the application may produce one or more graphical user interfaces (GUIs) which display pain levels; alpha, beta, gamma, delta, mu and theta waves; indications of facial movement; meditation and attention applications and treatment programs that help the patient reduce their perception of pain (as will be described further below).

FIG. 8 illustrates an example pain feedback device with a plurality of pain indicator ears, according to some embodiments. The pain feedback headset device is similar to that described in FIGS. 5A and 5B, but is equipped with a visual indicator of a level of pain in the form of a pair of bendable ears. The ears are configured to change shape and position depending on the level of pain that the patient is experiencing. For example, ears down when the patient is detected that he or she is not in pain, ears up mean he or she is detected to be in pain. The ears are only an example of a device based on a targeted patient group (e.g., targeted for the pediatric market). Any set of indicators may be implemented. For example, another simple expression of pain can be indicated using light, such as “on” means in pain and “off” means not in pain. Another example is using colors, such as “green,” “yellow,” and “red” for indicating not in pain, somewhat in pain, and in pain, respectively.

Thus, the pain feedback headset device is configured to receive assessments of the level of pain from the mobile device or server, or has the ability to calculate these levels on its own. FIG. 9 illustrates different example positions of the pain indicator ears of FIG. 8 as they correspond to different levels of pain, according to some embodiments. For example, if the ears lie flat, the patient may not be experiencing any pain, whereas if the ears are standing straight up, the patient may be experiencing a significant amount of pain. In some implementations, there may be different number of intermediate positions for indicating intermediate levels of pain.

FIG. 10 illustrates the different example positions of the pain indicator ears of FIG. 9 as they correspond to varying levels of mental stress, relaxation and pain, according to some embodiments. Once the pain detection algorithm is computed, any EEG data can be utilized to determine a pain level without requiring additional input from the patient.

An application and explanation of the correlation between mental relaxation, mental stress and pain is illustrated in FIG. 10 with regard to the varying levels of mental stress, relaxation and pain, according to some embodiments. FIG. 10 specifically illustrates how pain is a function of mental relaxation. As illustrated in FIG. 10, rising levels of pain correspond to decreased levels of relaxation. Mental stress is also increased with increased pain. As the amount of pain decreases, mental stress similarly decreases, and at a certain point, the brain begins to experience relaxation again. It is this correlation between pain and mental relaxation that provides unique insight into methods for assessing pain as a function of mental relaxation and creating therapies and treatments to decrease mental stress and increase relaxation to dull the effects of pain.

II. Treating Pain, Discomfort and Anxiety

By automatically and accurately determining a level of pain being experienced by a patient, the treatment of that pain may be uniquely tailored. If the pain rises only to a moderate level that would be considered discomfort or anxiety, the patient may be given techniques to simply reduce their perception of pain and decrease their overall mental stress, similarly to the chart illustrated in FIG. 10. By more accurately evaluating the levels of pain experienced by the patients, the healthcare provider can avoid prescribing unnecessary pain medication or under-prescribing medication and having the patient experience unnecessary pain.

Diversionary Therapy

One technique for reducing the perception of pain in a user is known as diversionary therapy (DT). With DT, the user is asked to complete a task or go through a series of activities or steps that are designed to mentally relax the user or have the user focus in order to reduce the effects of pain. Hospitals and other healthcare facilities are adopting DT as preliminary steps in pain assessment in order to reduce the amount of pain medication being dosed and help patients better deal with their pain and perception of pain.

The pain assessment algorithm may be applied to evaluate DT techniques and determine if they are effective in reducing the patient's perception of pain. Thus, the systems and methods of assessing pain levels described herein may be highly effective components of DT that will further increase the use and effectiveness of DT.

Applications of Pain and Discomfort Detection

In some embodiments, the patient may be presented with an interactive graphical interface with a picture representing their level pain or discomfort, such as storm clouds. As the patient's discomfort or pain levels increase, the storm clouds will increase, and as the patient's stress, anxiety, discomfort or pain decrease, the storm clouds will decrease. This activity may allow the user to focus on how to mentally reduce their pain, discomfort, stress or anxiety in real-time, allowing the patient to learn the mental and physical techniques that correlate to this reduction

In some embodiments, the EEG signals may also be correlated to coughing, so that the system can detect when a user is coughing. The system may then be configured to measure the amount of coughing and provide an alert if the coughing reaches a level that would warrant attention by a healthcare provider.

Reducing Anxiety and Stress

Similarly to the process of diversionary therapy (DT) described above, the pain assessment algorithm may also be utilized to measure a patient's stress or anxiety levels, which may also be determined by analyzing EEG signal data or facial expressions. In some embodiments, a patient experiencing stress or anxiety may be provided with an application or activity running on the mobile device which is designed to help the user reduce their stress or anxiety. The pain assessment algorithm may then be utilized to measure the effect of the activity on the patient, and may even use the measured data as a real-time component of the activity.

FIG. 11 shows an example of a process implementation. Process 1100 may be implemented using a device (e.g., device 104) to execute an algorithm that, at block 1110, receives electroencephalography (EEG) signals from an EEG sensor (e.g., EEG sensor 102). The EEG signals may be received for a time window (e.g., five seconds, 10 seconds, or other length of time). At block 1120, device 104 may determines, using correlated EEG data, a pain level based on the received EEG signals. The correlated EEG data, described in FIGS. 2-4 above, is EEG data created by, for example, correlating (e.g., associating, assigning, or marking) sample EEG signals with two or more pain level indicators. For example, sample EEG signals of certain characteristics (e.g., based on alpha waves and/or beta waves) may be correlated or associated with a pain level of high. Sample EEG signals of another set of characteristics may be correlated or associated with a pain level of low. Sample EEG signals of other sets of characteristics may be correlated or associated with pain levels in between high and low. At block 1130, when a pain level has been computed, identified, looked up, or otherwise determined, the pain level's indicator (e.g., high, low, etc.) may be presented or displayed on the device 104. The pain level may also be transmitted to a health service provider, for example.

In some embodiments, the correlated EEG data may be produced by correlating sample EEG signals with two or more sample pain levels (e.g., using stimulators). For example, a process for generating the correlated EEG data, which may be used to detect pain levels on one patient, a few patients, many patients, or all patients, may be include the operations of:

Obtaining sample EEG signals from a sample person or animal (participant). The sample EEG signals may be obtained over a period of time, such as five seconds, 10 seconds, or another interval. The sample EEG signals may be analyzed to determine the alpha waves and beta waves characteristics;

Administering a stimulated pain level using, for example, a negative external stimulus;

Computing an association of the characteristics of the sample EEG signals with the stimulated pain level; and

Recording the association of the characteristics of the sample EEG signals with the stimulated pain level.

The operations from obtaining to recording described above may be repeated with different sample EEG signals from the same participant and different stimulated pain levels. The operations may also be repeated with different participants (e.g., to create correlated EEG data useful on more than one patient).

In some examples, process 1100 may be implemented with different, fewer, or more blocks. Process 1100 may be implemented as computer executable instructions, which can be stored on a medium, loaded onto one or more processors of one or more computing devices, and executed as a computer-implemented method.

III. Computer Embodiments

FIG. 12 shows another example computing environment with an example device suitable for use in some example implementations. Device 1205 in computing environment 1200 can include one or more processing units, cores, or processors 1210, memory 1215 (e.g., RAM, ROM, and/or the like), internal storage 1220 (e.g., magnetic, optical, solid state storage, and/or organic), and/or I/O interface 1225, any of which can be coupled on a communication mechanism or bus 1230 for communicating information or embedded in the device 1205. Device 1205 may represent device 104 and/or server 106 (FIG. 1).

Device 1205 (may be referred to as a computing device) can be communicatively coupled to input/user interface 1235 and output device/interface 1240. Either one or both of input/user interface 1235 and output device/interface 1240 can be a wired or wireless interface and can be detachable. Input/user interface 1235 may include any device, component, sensor, or interface, physical or virtual, that can be used to provide input (e.g., buttons, touch-screen interface, keyboard, a pointing/cursor control, microphone, camera, braille, motion sensor, optical reader, and/or the like). Output device/interface 1240 may include a display, television, monitor, printer, speaker, braille, or the like. In some example implementations, input/user interface 1235 and output device/interface 1240 can be embedded with or physically coupled to the computing device 1205. In other example implementations, other computing devices may function as or provide the functions of input/user interface 1235 and output device/interface 1240 for a computing device 1205.

Examples of computing device 1205 may include, but are not limited to, highly mobile devices (e.g., smartphones, devices in vehicles and other machines, devices carried by humans and animals, and the like), mobile devices (e.g., tablets, notebooks, laptops, personal computers, portable televisions, radios, and the like), and devices not designed for mobility (e.g., desktop computers, other computers, information kiosks, televisions with one or more processors embedded therein and/or coupled thereto, radios, and the like).

Computing device 1205 can be communicatively coupled (e.g., via I/O interface 1225) to external storage 1245 and network 1250 for communicating with any number of networked components, devices, and systems, including one or more computing devices of the same or different configuration. Computing device 1205 or any connected computing device can be functioning as, providing services of, or referred to as a server, client, thin server, general machine, special-purpose machine, or another label.

I/O interface 1225 can include, but is not limited to, wired and/or wireless interfaces using any communication or I/O protocols or standards (e.g., Ethernet, 802.11x, Universal System Bus, WiMax, modem, a cellular network protocol, and the like) for communicating information to and/or from at least all the connected components, devices, and network in computing environment 1200. Network 1250 can be any network or combination of networks (e.g., the Internet, local area network, wide area network, a telephonic network, a cellular network, satellite network, and the like).

Computing device 1205 can use and/or communicate using computer-usable or computer-readable media, including transitory media and non-transitory media. Transitory media include transmission media (e.g., metal cables, fiber optics), signals, carrier waves, and the like. Non-transitory media include magnetic media (e.g., disks and tapes), optical media (e.g., CD ROM, digital video disks, Blu-ray disks), solid state media (e.g., RAM, ROM, flash memory, solid-state storage), and other non-volatile storage or memory.

Computing device 1205 can be used to implement techniques, methods, applications, processes, or computer-executable instructions in some example computing environments. Computer-executable instructions can be retrieved from transitory media, and stored on and retrieved from non-transitory media. The executable instructions can originate from one or more of any programming, scripting, and machine languages (e.g., C, C++, C#, Java, Visual Basic, Python, Perl, JavaScript, and others).

Processor(s) 1210 can execute under any operating system (OS) (not shown), in a native or virtual environment. One or more applications can be deployed that include logic unit 1260, application programming interface (API) unit 1265, input unit 1270, output unit 1275, training unit 1280, EEG sensor manager 1285, assessment/treatment 1290, and inter-unit communication mechanism 1295 for the different units to communicate with each other, with the OS, and with other applications (not shown). For example, training unit 1280, EEG sensor manager 1285, and assessment/treatment 1290 may implement one or more processes described and/or shown in FIGS. 1-11. The described units and elements can be varied in design, function, configuration, or implementation and are not limited to the descriptions provided.

In some example implementations, when information or an execution instruction is received by API unit 1265, it may be communicated to one or more other units (e.g., logic unit 1260, input unit 1270, output unit 1275, training unit 1280, EEG sensor manager 1285, and assessment/treatment 1290). For example, training unit 1280, along with other units 1260-1275, may perform some of the operations described in FIGS. 2-4 to trained or create correlated EEG data. When an EEG sensor attached to device 1205 transmits EEG signals to device 1205, the signals travel to I/O interface 1225, to input unit 1270, which passes the signals to EEG management EEG sensor manager 1285. EEG sensor manager 1285 interacts with, for example, training Unit 1280 to access the correlated EEG data to made an assessment based on the received EEG data. In some implementations, pain levels assessment (e.g., a pain level has been determined based on the EEG signals) based on the received EEG signals may be performed by assessment/treatment unit 1290, which may be executed to assess and/or treat a pain.

In some instances, logic unit 1260 may be configured to control the information flow among the units and direct the services provided by API unit 1265, input unit 1270, output unit 1275, training unit 1280, EEG sensor manager 1285, and assessment/treatment 1290 in some example implementations described above. For example, the flow of one or more processes or implementations may be controlled by logic unit 1260 alone or in conjunction with API unit 1265.

The I/O interface 1225 includes optional wireless communication components (not shown) that facilitate wireless communication over a voice and/or over a data network. The wireless communication components may include an antenna system, a radio system, a baseband system, or any combination thereof. Radio frequency (RF) signals may be transmitted and received over the air by the antenna system under the management of the radio system.

In some embodiments, the antenna system may include one or more antennae and one or more multiplexors that perform a switching function to provide the antenna system with transmit and receive signal paths. In the receive path, received RF signals can be coupled from a multiplexor to a low noise amplifier that amplifies the received RF signal and sends the amplified signal to the radio system.

In alternative embodiments, the radio system may comprise one or more radios that are configured to communicate over various frequencies. In some embodiments, the radio system may combine a demodulator and modulator in one integrated circuit (IC). The demodulator and modulator can also be separate components. In the incoming path, the demodulator strips away the RF carrier signal leaving a baseband receive audio signal, which is sent from the radio system to the baseband system.

If the received signal contains audio information, then baseband system decodes the signal and converts it to an analog signal. Then the signal is amplified and sent to a speaker. The baseband system also receives analog audio signals from a microphone. These analog audio signals are converted to digital signals and encoded by the baseband system. The baseband system also codes the digital signals for transmission and generates a baseband transmit audio signal that is routed to the modulator portion of the radio system. The modulator mixes the baseband transmit audio signal with an RF carrier signal generating an RF transmit signal that is routed to the antenna system and may pass through a power amplifier. The power amplifier amplifies the RF transmit signal and routes it to the antenna system where the signal is switched to the antenna port for transmission.

Any of the software components described herein may take a variety of forms. For example, a component may be a stand-alone software package, or it may be a software package incorporated as a “tool” in a larger software product. It may be downloadable from a network, for example, a website, as a stand-alone product or as an add-in package for installation in an existing software application. It may also be available as a client-server software application, as a web-enabled software application, and/or as a mobile application.

Although a few example implementations have been shown and described, these example implementations are provided to convey the subject matter described herein and to enable any person skilled in the art to make or use the subject matter described herein. It should be understood that the subject matter described herein may be implemented in various forms without being limited to the described example implementations. The subject matter described herein can be practiced without those specifically defined or described matters or with other or different elements or matters not described. It will be appreciated by those familiar with this field that changes may be made in these example implementations without departing from the subject matter described herein as defined in the appended claims and their equivalents. Thus, the subject matter discussed herein is not intended to be limited to the embodiments or examples described herein, but is to be accorded the widest scope consistent with the principals and novel features disclosed herein.

What is claimed is:

1. A method of assessing a pain level of a patient, comprising:
receiving within a time window, using a device, electroencephalography (EEG) signals of the patient from an EEG sensor;
determining, using the device and correlated EEG data, a pain level based on the EEG signals of the patient, wherein the correlated EEG data is created by correlating other EEG signals with two or more pain level indicators; and
presenting, on a screen of the device or another device, one of the pain level indicators.

2. The method of claim 1, wherein the time window is about 10 seconds.

3. The method of claim 1, wherein the determining the pain level based on the EEG signals comprising determining the characteristics of alpha waves and beta waves of the EEG signals.

4. The method of claim 1, wherein the correlating other EEG signals with two or more pain level indicators comprising:
measuring a first set of characteristics of alpha waves and beta waves in a first portion of the other EEG signal over a first time interval;
applying a first negative external stimulus that represents a first pain level;
associating the first set of characteristics with the first pain level;
recording the association of the first pain level with the first set of characteristics; and
repeating, at least once:
measuring a next set of characteristics of alpha waves and beta waves in a next portion of the other EEG signal over a next time interval;
applying a next negative external stimulus that represents a next pain level;
associating the next set of characteristics with the next pain level; and
recording the association of the next pain level with the next set of characteristics.

5. The method of claim 1, wherein the presenting the one of the pain level indicators is on the another device which is a pain feedback device.

6. The method of claim 1, further comprising applying a diversionary therapy to reduce the pain level.

7. The method of claim 1, wherein the device is a mobile device.

8. A non-transitory computer readable medium having stored therein computer executable instructions for:
receiving within a time window, using a device, electroencephalography (EEG) signals of a patient from an EEG sensor;
determining, using the device and correlated EEG data, a pain level based on the EEG signals of the patient, wherein the correlated EEG data is created by correlating other EEG signals with two or more pain level indicators; and
presenting, on a screen of the device or another device, one of the pain level indicators.

9. The computer readable medium of claim 8, wherein the time window is about 10 seconds.

10. The computer readable medium of claim 8, wherein the determining the pain level based on the EEG signals comprising determining the characteristics of alpha waves and beta waves of the EEG signals.

11. The computer readable medium of claim 8, wherein the correlating other EEG signals with two or more pain level indicators comprising:
measuring a first set of characteristics of alpha waves and beta waves in a first portion of the other EEG signal over a first time interval;
applying a first negative external stimulus that represents a first pain level;
associating the first set of characteristics with the first pain level;
recording the association of the first pain level with the first set of characteristics; and
repeating, at least once:
measuring a next set of characteristics of alpha waves and beta waves in a next portion of the other EEG signal over a next time interval;
applying a next negative external stimulus that represents a next pain level;
associating the next set of characteristics with the next pain level; and
recording the association of the next pain level with the next set of characteristics.

12. The computer readable medium of claim 8, wherein the presenting the one of the pain level indicators is on the another device which is a pain feedback device.

13. The computer readable medium of claim 8, further comprising instructions for applying a diversionary therapy to reduce the pain level.

14. At least one computing device comprising storage and a processor configured to perform:
receiving within a time window, using a device, electroencephalography (EEG) signals of a patient from an EEG sensor;
determining, using the device and correlated EEG data, a pain level based on the EEG signals of the patient, wherein the correlated EEG data is created by correlating other EEG signals with two or more pain level indicators; and
presenting, on a screen of the device or another device, one of the pain level indicators.

15. The least one computing device of claim 14, wherein the time window is about 10 seconds.

16. The least one computing device of claim 14, wherein the determining the pain level based on the EEG signals comprising determining the characteristics of alpha waves and beta waves of the EEG signals.

17. The least one computing device of claim 14, wherein the correlating other EEG signals with two or more pain level indicators comprising:
measuring a first set of characteristics of alpha waves and beta waves in a first portion of the other EEG signal over a first time interval;
applying a first negative external stimulus that represents a first pain level;
associating the first set of characteristics with the first pain level;
recording the association of the first pain level with the first set of characteristics; and
repeating, at least once:
measuring a second set of characteristics of alpha waves and beta waves in a second portion of the other EEG signal over a second time interval;
applying a second negative external stimulus that represents a second pain level;
associating the second set of characteristics with the second pain level; and
recording the association of the second pain level with the second set of characteristics.

18. The least one computing device of claim 14, wherein the presenting the one of the pain level indicators is on the another device which is a pain feedback device.

19. The least one computing device of claim 14, wherein the processor is further configured to perform applying a diversionary therapy to reduce the pain level.

20. The least one computing device of claim 14, wherein the device is a mobile device.
